(19) United States
(12) Patent Application Publication
(10) Pub. No.: US 2007/0250104 A1
(43) Pub. Date: Oct. 25, 2007

(54) Catheter and Method for Dilating a Body Passageway

(75) Inventors: Alexander Condrea, Haifa (IL); Arie Vadasz, Haifa (IL); Eliran Chiel, Shimshit (IL)

Correspondence Address: NATH & ASSOCIATES, 112 South West Street, Alexandria, VA, 22314, US

(73) Assignee: MEDILATOR, Migdal Emek (IL)

(21) Appl. No.: 10/592,065

(22) PCT Filed: Mar. 8, 2005
(86) PCT No.: PCT/IL05/00271

§ 371 Date: Mar. 26, 2007

Related U.S. Application Data
(60) Provisional application No. 60/550,729, filed on Mar. 8, 2004.
(60) Provisional application No. 60/621,469, filed on Oct. 25, 2004.
(60) Provisional application No. 60/627,896, filed on Nov. 16, 2004.

(51) Int. Cl. A61M 29/02

(52) U.S. Cl. 606/193

(57) ABSTRACT

The invention provides catheter for expanding a body passageway leading to a body cavity, such as a cervix leading to a uterus. The catheter comprises an inflatable balloon located at the distal end of the catheter. The balloon has an anterior portion and a posterior portion. The anterior portion is inflatable prior to inflation of the posterior portion. The invention also provides a system for inflating a body passageway leading to a body cavity comprising the catheter

### FIELD OF THE INVENTION

This invention relates to devices for dilating a body passageway such as a cervix.

### BACKGROUND OF THE INVENTION

There are several situations necessitating the dilation of a body passage way, such as a cervix. For example, it is known to insert into the cervix a series of insertion devices of increasing caliber to achieve a gradual dilation of the cervix. It is also known to insert an absorbent material into the cervix. The material is allowed to absorb a body liquid or other liquid so as to expand, thus dilating the cervix.

It is also known to achieve dilation of the cervix by inserting an uninflated balloon into the cervix and inflating the balloon. Such devices are disclosed, for example, in U.S. Pat. No. 4,664,114 to Ghodsian, U.S. Pat. No. 3,900,033 to Leininger et al, U.S. Pat. No. 4,089,337, to Kronser, and U.S. Pat. No. 4,490,421 to Levy.

### SUMMARY OF THE INVENTION

In its first aspect, the present invention provides a system for dilating a body passageway leading to a body cavity. The body passageway may be, for example, a cervix and the body cavity a uterus. The system of the invention comprises a catheter and a control unit 4. The catheter comprises an elongated shaft having a proximal end connected to the control unit. At the distal end of the shaft is an inflatable balloon. The shaft may be flexible or rigid, as required in any application. The shaft comprises at least one lumen for delivering a pressurized fluid from the control unit to the balloon for inflating the balloon in order to inflate the passageway. The balloon has an anterior portion and a posterior portion. In accordance with the invention, the pressurized fluid is delivered from the control unit to the balloon via the lumen or lumens of the shaft so as to inflate the anterior portion of the balloon prior to inflation of the posterior portion. The balloon may be of various dimensions (length, diameter, etc.) and shapes (short conical, elongated conical, elliptical, spherical combinations) as required in any application. The balloon may be made from an elastic material which stretches as the balloon is inflated. Alternatively, the balloon may be made from an inelastic flexible material in which case the balloon has a defined maximal volume to which it can be inflated.

In its second aspect, the invention provides a catheter for use in the system of the invention.

In its third aspect, the invention provides a method for dilating a body passageway leading to a body cavity. The body passageway may be, for example, a cervix and the body cavity a uterus. In one embodiment of the method of the invention, the distal end of the catheter is introduced into the body passageway with the balloon uninflated and folded around the shaft. The distal end of the catheter is positioned with the anterior portion in the body cavity. Pressurized fluid is then delivered to the anterior portion only so as to inflate the anterior portion in the body cavity to a diameter that exceeds the diameter of the passageway. After inflation of the anterior portion is completed, the catheter is displaced proximally so as to lodge the inflated anterior portion at the opening of the passageway into the cavity. The anterior portion may be deflated at this point and pressurized fluid delivered to the posterior portion so as to inflate the posterior portion with the anterior portion deflated, or the pressurized fluid may be delivered to the posterior portion to inflate the posterior portion with the anterior portion inflated. As the posterior portion is inflated, the body passageway is dilated.

In another embodiment of the method of the invention, only the anterior portion of the catheter is introduced into a first portion of the passageway with the balloon uninflated and wrapped around the shaft. The anterior portion is then inflated to dilate that specific part of the passageway. Subsequently, the anterior portion is deflated and the catheter is introduced further into the. Then the anterior portion is inflated once again to dilate that specific part of the passageway. The anterior portion is then deflated and the process is repeated until the anterior portion has reached end of the passageway. Then either the entire balloon just the posterior portion of the balloon is inflated to dilate the passageway to a final diameter. The balloon is then deflated and catheter is removed from the passageway.

Thus, in its first aspect, the invention provides a catheter for expanding a body passageway leading to a body cavity, comprising:
(a) an elongated shaft having a proximal end and a distal end, a wall and at least one lumen; and
(b) an inflatable first balloon located at the distal end of the shaft having an anterior portion and a posterior portion;
   wherein the anterior portion is inflatable prior to inflation of the posterior portion.

In its second aspect, the invention provides a system for inflating a body passageway leading to a body cavity, comprising:
(a) A catheter of the invention having a first socket on the proximal end of the shaft; and
(b) A control unit having a second socket mating with the first socket; the control unit providing pressurized fluid to a lumen of the shaft when the first socket is attached t the second socket.

In its third aspect, the invention provides a method for dilating a body passageway leading to a body cavity, comprising:
(a) inserting the distal end of a catheter of the invention into the passageway with the first balloon in an uninflated state;
(b) positioning the anterior portion of the first balloon inside the body cavity;
(c) inflating the anterior portion of the first balloon in the body cavity;
(d) displacing the distal end proximally so as to lodge the inflated anterior portion in an opening of the passageway into the body cavity; and
(e) inflating the posterior portion in the passageway so as to expand the passageway.

In its fourth aspect, the invention provides a method for dilating a body passageway leading to a body cavity, comprising:
(a) delivering the anterior portion of a catheter according to any one of Claims 1 to 28 to a segment of the passageway with the first balloon in an uninflated state;
(b) inflating the anterior portion of the first balloon in the segment;
(c) displacing the distal end proximally;
(d) inflating the posterior portion in the passageway; and
(e) repeating steps (b) to (d) as required to achieve a desired dilation of the body passageway.

In its fifth aspect, the invention provides method for determining whether the resistance of a wall of a body passageway at a first location in the passageway is normal comprising:
(a) inserting a distal end of a catheter of the invention into the passageway;
(b) positioning the first balloon at the first location in the passageway;
(c) inflating the balloon; and
(d) determining whether an external pressure exerted by the passageway wall on the inflated balloon is above a predetermined threshold;
   an external force exerted on the balloon above the predetermined threshold being indicative of a normal resistance.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a system for dilating a body passageway in accordance with one embodiment of the system of the invention;

FIG. 2 shows a catheter in accordance with one embodiment of the invention;

FIG. 3 shows the control unit of the system of FIG. 1;

FIG. 4 shows a catheter in accordance with another embodiment of the invention;,

FIG. 5 shows a catheter in accordance with another embodiment of the invention;

FIG. 6 shows a catheter in accordance with another embodiment of the invention;

FIG. 7 shows a catheter in accordance with another embodiment of the invention;

FIG. 8 shows a catheter in accordance with another embodiment of the invention;

FIG. 9 shows a catheter in accordance with another embodiment of the invention;

FIG. 10 shows a catheter in accordance with another embodiment of the invention;

FIG. 11 shows a catheter in accordance with another embodiment of the invention;

FIG. 12 shows a catheter in accordance with another embodiment of the invention;

FIG. 13 shows a catheter in accordance with another embodiment of the invention;

FIG. 14 shows a method for dilating a body passageway in accordance with one embodiment of the invention;

FIG. 15 shows a method for dilating a body passageway in accordance with another embodiment of the invention;

FIG. 16 shows a catheter in accordance with another embodiment of the invention; and

FIG. 17 shows a catheter in accordance with another embodiment of the invention;

### DETAILED DESCRIPTION OF PREFERRED EMBODIMENT OF THE INVENTION

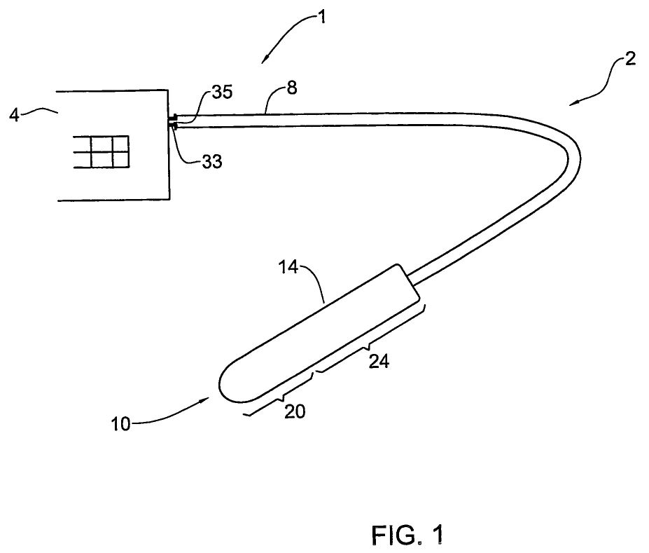

FIG. 1 shows a system 1 for dilating a body passageway leading to a body cavity in accordance with one embodiment of the invention. The system 1 comprises a catheter 2 and a control unit 4. The catheter 2, comprises an elongated shaft 6 having a proximal end 8 connected to the control unit 4 and a distal end 10. At the distal end 10 of the shaft 6 is an inflatable balloon 14. The shaft 6 may be flexible or rigid, as required in any application. The shaft 6 comprises at least one lumen for delivering a pressurized fluid from the control unit to the balloon 14 for inflating the balloon. The balloon 14 has an anterior portion 20 and a posterior portion 24. As explained below in relation to the various embodiments of the catheter 2, the pressurized fluid is delivered from the control unit 4 to the balloon 14 via the lumen or lumens of the shaft 6 so as to inflate the anterior portion 20 prior to inflation of the posterior portion. The anterior portion 20 and the posterior portion 24 may be adjacent to each other along the balloon 14, or may be separated by an intervening central region. The balloon 14 may be of various dimensions (length, diameter, etc.) and shapes (short conical, elongated conical, elliptical, spherical combinations) as required in any application. The balloon 14 may be made from an elastic material which stretches as the balloon 14 is inflated. Alternatively, the balloon 14 may be made from an inelastic flexible material in which case the balloon 14 has a defined maximal volume to which it can be inflated.

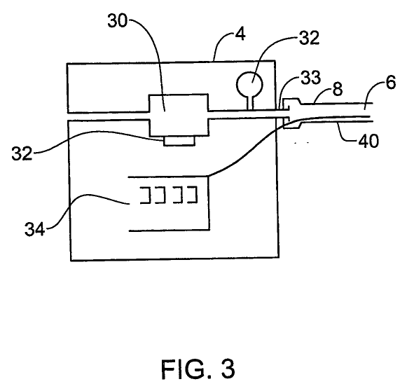

FIG. 3 schematically shows the control unit 4 in greater detail. The control unit 4 has a socket 33 that mates with a socket 35 at the proximal end 8 of the shaft 6, for attachment of the shaft 6 to the control unit 4. The control unit 4 includes a pressure generator 30 for pressurizing a fluid to be delivered to the distal end 10 of the catheter 2. The fluid may be a gas such as air, or may be a liquid such as water. The pressure generator 30 may be an electric pump located inside the control unit 4, as shown in FIG. 3. Alternatively, the pressure generator may be an electric pump located outside of the control unit 4, in order to avoid vibrations in the control unit 4. In this case, the pressure generator may be located remotely from the control unit 4. For example, the control unit may be connected via a tube to a tap of a pressurized fluid such as compressed air or water. The pressure generator may also be a manually or foot-operated pump.

The control unit 4 also includes a pressure gauge 32 for monitoring the pressure of the pressurized fluid. The pressure of the fluid may be displayed on a digital display 34. The control unit 4 may also comprise a regulated heating unit 36 for heating the fluid to a selectable temperature before flowing into the catheter 2. The selected temperature and/or the actual temperature of the fluid may be displayed on the digital display 34.

The controller may also be provided with an alarm that generates a sensible signal when the fluid pressure or fluid temperature exceeds a pre-set level. The alarm may be, for example, an audio alarm or a visual alarm, such as a flashing light.

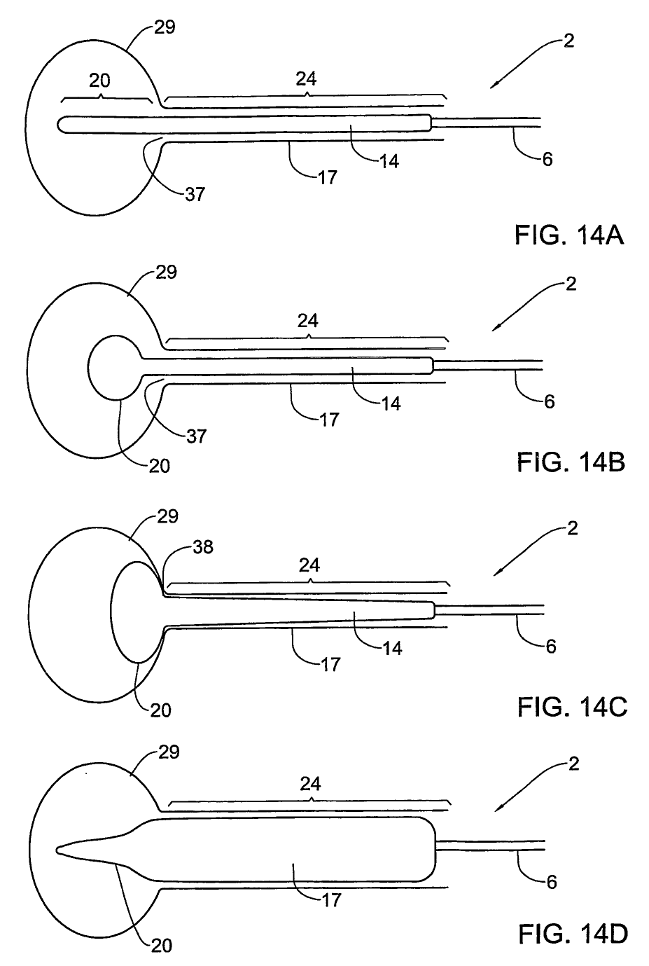

FIG. 14 shows use of the system of the invention for dilation of a body passageway 17 leading to a body cavity 29 in accordance with one embodiment of the method of the invention. The body passageway 17 may be, for example, a cervix and the body cavity 29 a uterus. As shown in FIG. 14a, the distal end 10 of the catheter 2 is introduced into the body passageway 17 with the balloon 14 uninflated and folded around the shaft 6. The distal end of the catheter 2 is positioned with the anterior portion 20 in the body cavity 29 to which the passageway 17 leads. Pressurized fluid is then delivered from the control unit 4 to the anterior portion 20 through one or more lumens in the shaft 6 so as to inflate the anterior portion 20 as shown in FIG. 14b. The anterior portion 20 is inflated in the body cavity 29 to a diameter that exceeds the diameter of the body passageway 17. After inflation of the anterior portion 20 is completed, the catheter 2 is displaced proximally so as to lodge the inflated anterior portion 20 at the opening 37 of the passageway 17 into the cavity 29 as shown in FIG. 14c. The anterior portion 20 may be deflated at this point and pressurized fluid delivered to the posterior portion 24 from the control unit 4, so as to inflate the posterior portion 24 with the anterior portion 20 deflated, as shown in FIG. 14d. Alternatively, pressurized fluid is delivered to the posterior portion 24 so as to inflate the posterior portion 24 with the anterior portion 20 inflated. As the posterior portion 24 is inflated, the body passageway 17 is dilated. Inflation of the posterior portion 24 is terminated when the passageway 17 has attained a predetermined dilation, or when a predetermined external pressure is exerted on the posterior portion 24 by the wall of the passageway 17. The external pressure may be determined from the pressure of the pressurized fluid (as registered by the pressure gauge 32) and by the amount of the fluid that has been introduced into the balloon 14. Inflation of the posterior portion 24 may be done in a gradual fashion with small increments of inflation. The catheter 2 may be pushed distally so that the distal end of the posterior portion 24 is located in the opening 37 before the posterior portion 24 is inflated. By doing so, the entire passageway 17, including the opening 37 is dilated as the posterior portion 24 is inflated.

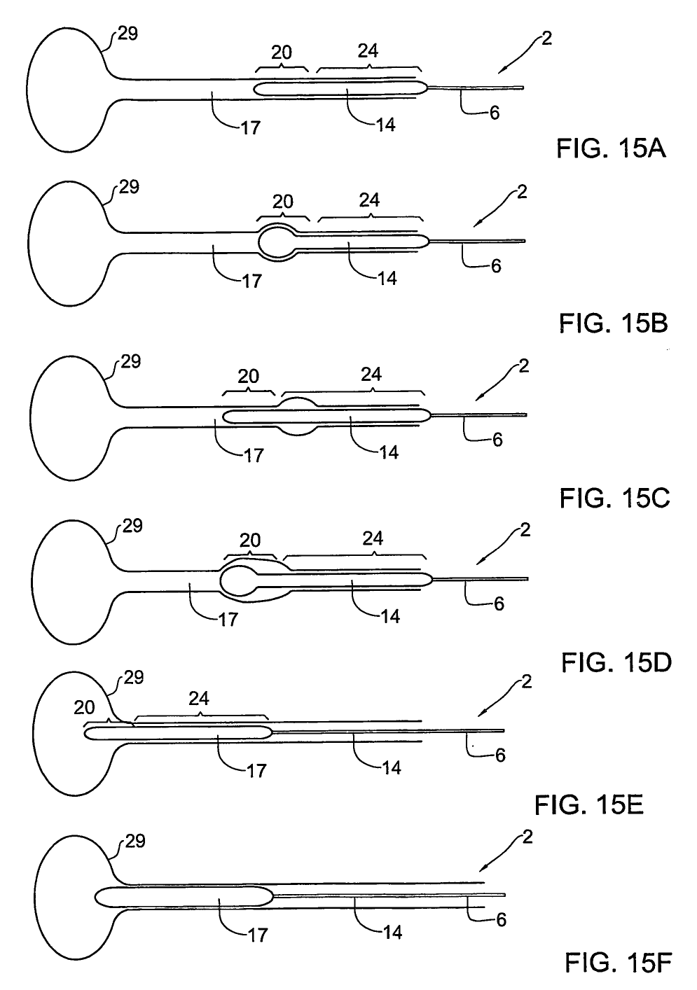

In another embodiment of the method of the invention, the anterior portion 20 of the catheter 2 is introduced into a first portion of the passageway 17 with the balloon 14 uninflated and wrapped around the shaft 6, as shown in FIG. 15a. Then the anterior portion 20 is inflated as shown in FIG. 15b. As a result, that specific part of the passageway 17 is dilated. Subsequently, the anterior portion 20 is deflated and the catheter 2 is introduced further into the passageway 17, for a distance about equal to the length of the anterior portion 20 (FIG. 15c). Then the anterior portion 20 is inflated once again (FIG. 15d). As a result, that specific part of the passageway is dilated. The anterior portion 20 is then deflated and the process is repeated until the anterior portion 20 has reached end of the passageway 17 (FIG. 15e). Then either the entire balloon 14 is inflated or just the posterior portion 24 of the balloon 14 is inflated. The result is a dilation of the passageway 17 to a final diameter.

Then the balloon 14 is deflated and catheter 2 is removed from the passageway 17.

In one embodiment of the method of the invention, a catheter 2 is first used having a small caliber, in order to obtain a first dilation of the passageway 17 in order to prepare the passageway 17 to receive larger caliber catheter 2 for dilating the passageway 17 to a final dilation. In this case, the final inflated caliber of the small caliber catheter should be around the initial uninflated caliber of the large caliber catheter.

### First Embodiment

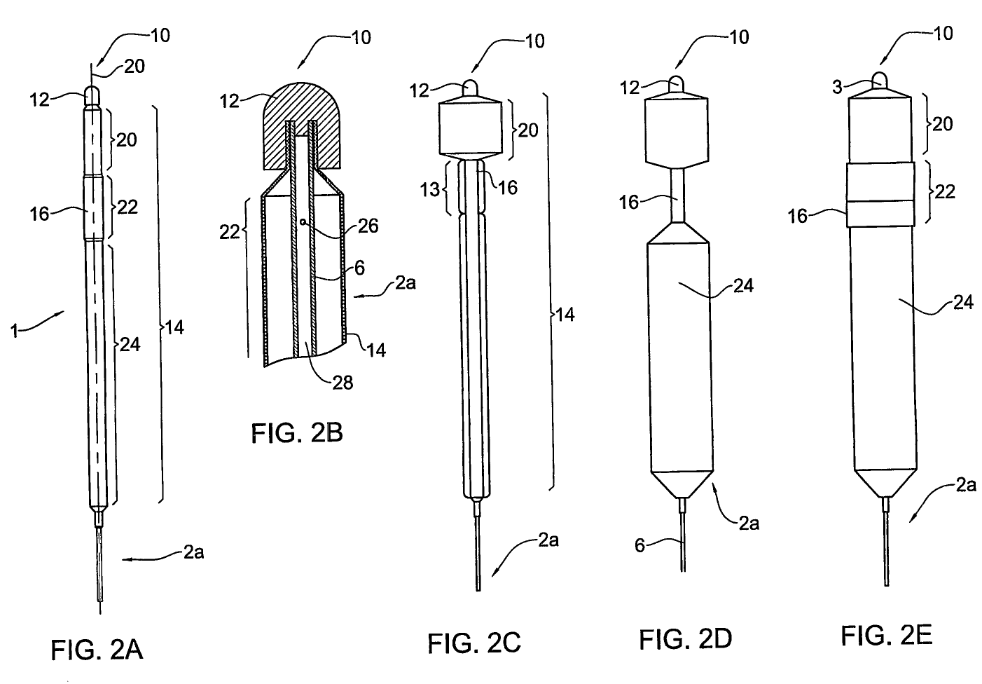

FIG. 2 shows the distal end 10 of a catheter 2a that may be used for the catheter 2. At the tip of the distal end is a blunt protector cap 12 to prevent wounding of a subject during insertion of the distal end 10 into the passageway. FIG. 2a shows the distal end 10 of the catheter 2a in a perspective view with the balloon 14 collapsed onto the shaft 6. A constraining ring 16 surrounds the balloon 14 at a central section 22 along the longitudinal axis 20 of the balloon 14. The constraining ring 16 thus divides the balloon into three sections. The anterior portion 20, distal to the constraining ring 16, the central section 22 underlying the constraining ring 16, and the posterior portion 24 proximal to the constraining ring 16. FIG. 2b shows a cross section of the tip of the catheter 2a in greater detail. The shaft 6 is hollow and has a single lumen 28 extending from the control unit 4 to the distal end 10 for conducting pressurized fluid from the control unit 4 to the distal end 10 of the catheter 2 as explained below. The pressurized fluid leaves the lumen 28 of the shaft 6 by means of one or more holes 26 located in the wall of the shaft 6. The one or more holes 26 are located only within the anterior portion 20 of the balloon 14. When pressurized fluid enters the anterior portion 20 through the hole or holes 26, the anterior portion 20 expands as shown in FIG. 2c. The central portion 22 at this point is prevented from expanding due to the presence of the constraining ring 16. The constraining ring 16 also prevents flow of the fluid from the anterior portion 20 to the posterior portion 24.

Once a first predetermined pressure has been attained in the anterior portion 20, the constraining ring 16 expands slightly, allowing the fluid to flow from the anterior portion 20 to the posterior portion 24, so that the balloon 14 attains the “dumbbell” shape shown in FIG. 2d. The constraining ring 16 may be made, for example, from silicone rubber. Once a second predetermined pressure has been attained in the entire balloon 14, the constraining ring 16 expands further until the balloon 14 has attained the cylindrical shape shown in FIG. 2e. Inflation of the anterior portion 20 thus occurs prior to inflation of the posterior portion 24.

The constraining ring 16 may be tethered to the shaft 6 in order to prevent the expanded constraining ring 16 from separating from the catheter after subsequent deflation of the balloon. This may be accomplished, for example, by means of a thin extension over the posterior portion 24, attaching the constraining ring 16 to the shaft 6 (not shown). The extension may be from the same material as the constraining ring 16, so as to allow the constraining ring 16 and the extension to be produced in a single molding operation.

### Second Embodiment

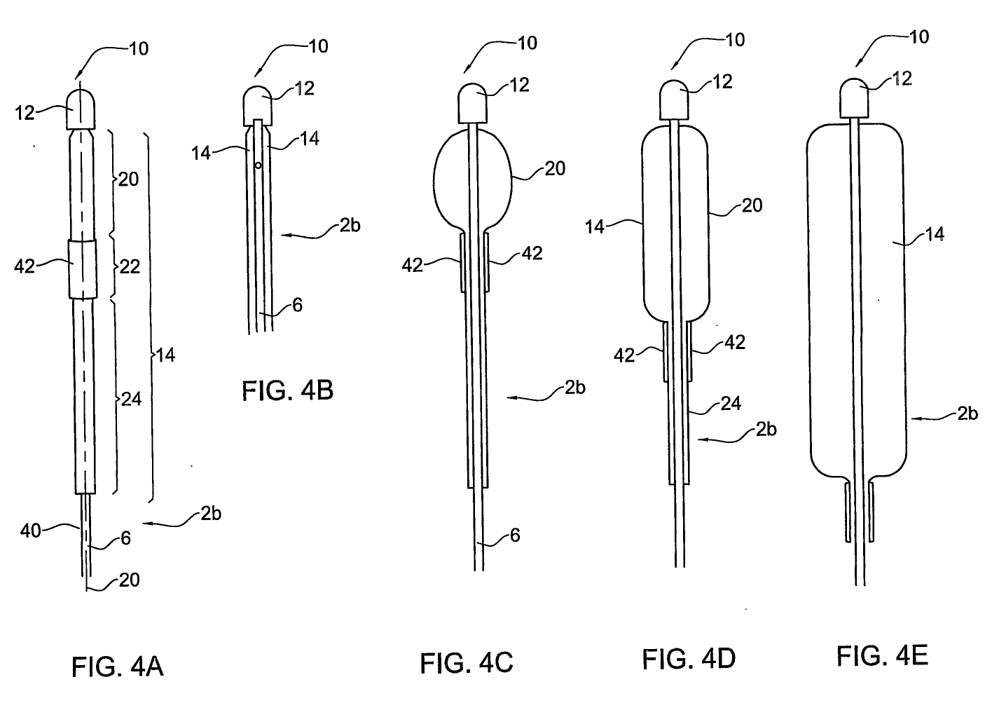

FIG. 4 shows the distal end 10 of a catheter 2b that may be used in the system 1 for the catheter 2. The catheter 2b has several elements in common with the catheter 2a, and similar elements are indicated by the same reference numeral without further comment.

FIG. 4a shows the distal end 10 of the catheter 2b in a perspective view with the balloon 14 collapsed onto the shaft 6. A constraining ring 42 surrounds the balloon 14 at a central section 44 along the longitudinal axis 20 of the balloon 14. The constraining ring 42 may be made, for example, from silicone rubber. The constraining ring 42 thus divides the balloon into three sections. The anterior portion 20, distal to the constraining ring 42, the central section 44 underlying the constraining ring 42, and the posterior portion 48 proximal to the constraining ring 42. FIG. 4b shows a cross section of the tip of the catheter 2b in greater detail. The pressurized fluid leaves the lumen 28 of the shaft 6 by means of the one or more holes 26 located in the wall of the shaft 6 that are located only within the anterior portion 20 of the balloon 14. When the pressurized fluid enters the anterior portion 20 through the hole or holes 26, the anterior portion 20 expands as shown in FIG. 4c. The central section 44 at this point is prevented from expanding due to the presence of the constraining ring 42. The constraining ring 42 also prevents flow of the fluid from the anterior portion 20 to the posterior portion 24.

As the anterior portion 20 expands, the constraining ring 42 slides proximally allowing a longer portion of the distal end of the balloon 14 to be inflated, as shown in FIG. 4d. This process continues until the constraining ring 42 has completely slid off the balloon 14 onto the exposed portion of the shaft 6, as shown in FIG. 4e, so that the posterior portion 24 of the balloon 14 is completely inflated. Inflation of the anterior portion 20 thus occurs prior to inflation of the posterior portion 24.

### Third Embodiment

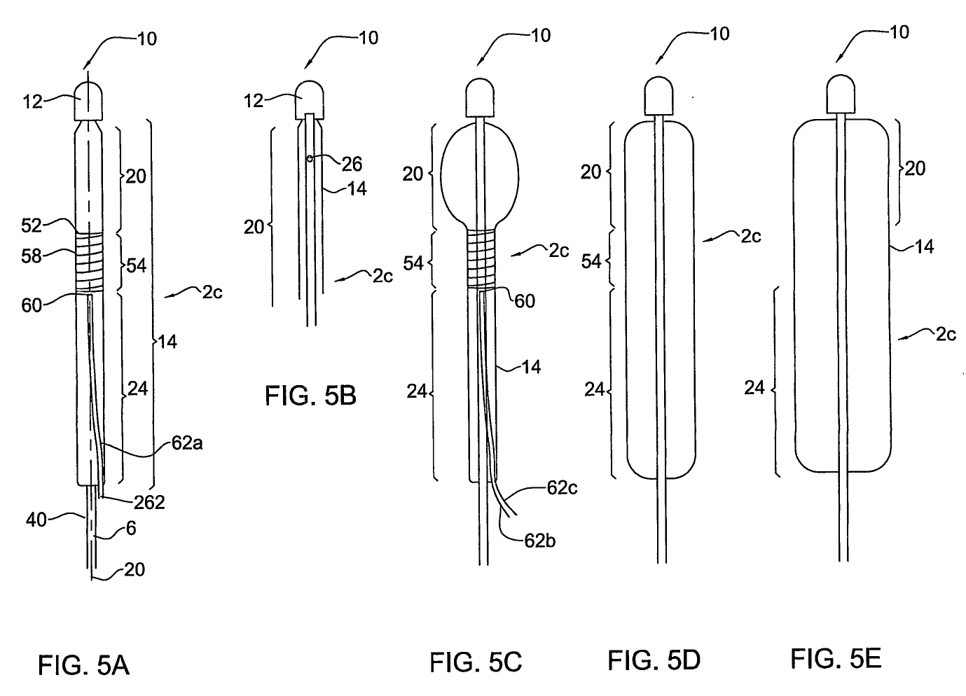

FIG. 5 shows the distal end 10 of a catheter 2c that may be used in the system 1 for the catheter 2. The catheter 2c has several elements in common with previously described embodiments of the catheter 2, and similar elements are identified by the same reference numeral without further comment.

FIG. 5a shows the distal end 10 of the catheter 2c in a perspective view with the balloon 14 collapsed onto the shaft 6. A constraining ring 52 surrounds the balloon 14 at a central section 54 along the longitudinal axis 20 of the balloon 14. The constraining ring 52 thus divides the balloon 14 into three sections. The anterior portion 20 distal to the constraining ring 52, the central section 54, and the posterior portion 24 proximal to the constraining ring 52.

The constraining ring 52 is formed from a filament 58 that is wrapped around the central region 54 of the balloon 14. The filament 58 may be formed from any biocompatible material used in the manufacture of filaments such as cotton or a metal. The filament 58 is tied in a slip-knot 60, and the ends 62a and 62b extend proximally along the length of the balloon 14 and the shaft 6 to the proximal end of the shaft 6 (not shown).

FIG. 5b shows a cross section of the tip of the catheter 2c in greater detail. Pressurized fluid leaves the lumen 24 of the shaft 6 by means of the one or more holes 26 located in the wall of the shaft 6 that are located only within the anterior portion 20 of the balloon 14. When pressurized fluid enters the anterior portion 20 through the hole or holes 26, the anterior portion 20 expands, as shown in FIG. 5c. The central section 58 at this point is prevented from expanding due to the presence of the constraining ring 52. The constraining ring 52 also prevents flow of the fluid from the anterior portion 20 to the posterior portion 24.

After the anterior portion 20 has been inflated to a predetermined size, the constraining ring is removed by pulling on one or both of the ends 62a and 62b of the filament 58. Pulling one or both of the ends 62a and 62b releases the slip-knot 60 so that the filament 58 may be removed. Removal of the constraining ring 58 allows the pressurized fluid previously restricted to the anterior portion 20 of the balloon 14 to enter the central section 54 and the posterior portion 24 of the balloon as indicated in FIG. 5d. Additional fluid may then be delivered to the balloon 14 until the balloon 14 is completely inflated as shown in FIG. 5e. Inflation of the anterior portion 20 thus occurs prior to inflation of the posterior portion 24.

### Fourth Embodiment

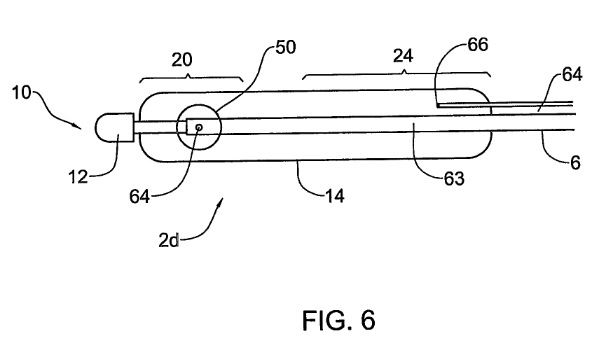

FIG. 6 shows the distal end 10 of a catheter 2d that may be used in the system 1 for the catheter 2. The catheter 2d has several elements in common with the previously described embodiments of the catheter 2 and similar elements are identified by the same reference numeral without further comment.

The distal end 10 of the catheter 2d comprises an inner balloon 50 located in the interior of the balloon 14 and confined to the anterior portion 20 of the balloon 14. Inflation of the inner balloon 50 expands the anterior portion of the balloon 14. The inner balloon 50 may be, for example, spherical in shape when inflated but it may have any shape (e.g. Cylindrical) and dimensions (various diameters and/or lengths) as well as compliant properties as required in any application.

The shaft 6 of the catheter 2d has two lumens 63 and 64. The lumen 63 conducts the pressurized fluid from the control unit 4 to the inner balloon 50 via a hole 64 in the wall of the shaft 6′. The second lumen 64 conducts the pressurized fluid from the control unit 4 to the posterior portion 24 of the balloon 14 balloon 14 via a hole 66 after inflation of the anterior portion 20. Inflation of the anterior portion 20 thus occurs prior to inflation of the posterior portion 24.

### Fifth Embodiment

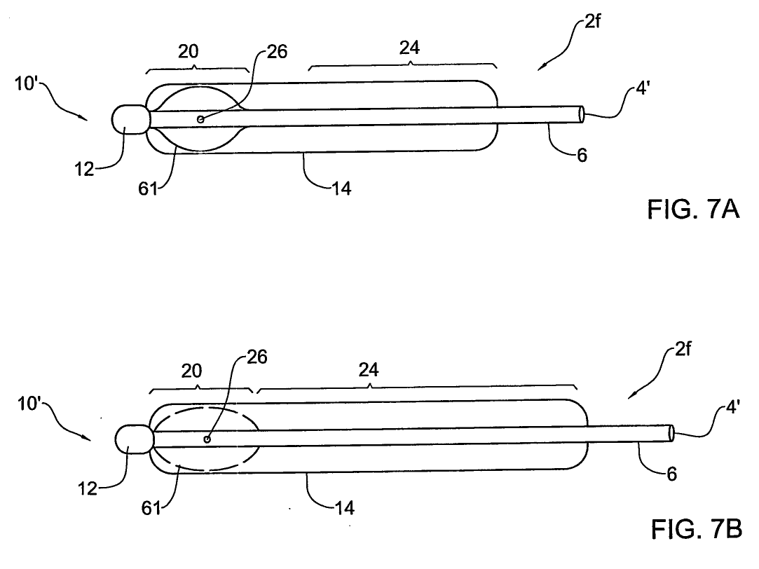

FIG. 7 shows the distal end of a catheter 2f that may be used in the system 1 for the catheter 2. The catheter 2f has several elements in common with the previously described embodiments of the catheter 2 and similar elements are identified by the same reference numeral without further comment.

In the catheter 2f, the shaft 6 delivers the pressurized fluid from the control unit 4 to the lumen of an inner balloon 61 through the hole 26. The inner balloon 61 is located in the interior of the balloon 14 and confined to the anterior portion 20 of the balloon 14. As shown in FIG. 7a, the pressurized fluid may be delivered to the lumen of the inner balloon 61 so as to inflate the inner balloon 61, thereby inflating the anterior portion 20 of the balloon 14. Pressurized fluid continues to be delivered to the inner balloon 60 until the pressure inside the first balloon exceeds a predetermined level at which the inner balloon 60 bursts.

When the inner balloon 61 bursts (FIG. 7b), the fluid previously contained in the lumen of the inner balloon 61 is released into the entire lumen of the balloon 14. Pressurized fluid continues to be delivered into the lumen of the balloon 14 from the control unit 4 through the lumen of the shaft 6 via the hole 26, so as to continue the inflation of the outer balloon 14. Inflation of the anterior portion 20 thus occurs prior to inflation of the posterior portion 24.

### Sixth Embodiment

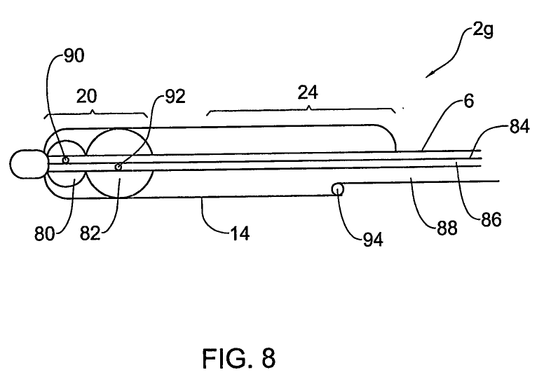

FIG. 8 shows the distal end 10 a catheter 2gthat may be used in the system 1 for the catheter 2. The catheter 2ghas several elements in common with the previously described embodiments of the catheter 2 and similar elements are identified by the same reference numeral without further comment.

Two inner balloons 80 and 82 are contained in the interior of the balloon 14 and confined to the anterior portion 20 of the balloon 14. The inner balloon 80 is smaller in diameter when inflated than the inner balloon 82. The catheter 2g has a shaft 6″ containing three lumens 84, 86 and 88. The lumen 84 delivers the pressurized fluid to the inner balloon 80 via a hole 90. The lumen 86 delivers the pressurized fluid to the inner balloon 82 via the hole 92. Sequential inflation of the inner balloon 80 followed by inflation of the inner balloon 82 provides controlled inflation of the anterior portion 20 of the balloon 14. Following inflation of the anterior portion 20 of the balloon 14, the posterior portion 24 may be inflated via the lumen 88. Inflation of the anterior portion 20 thus occurs prior to inflation of the posterior portion 24.

### Seventh Embodiment

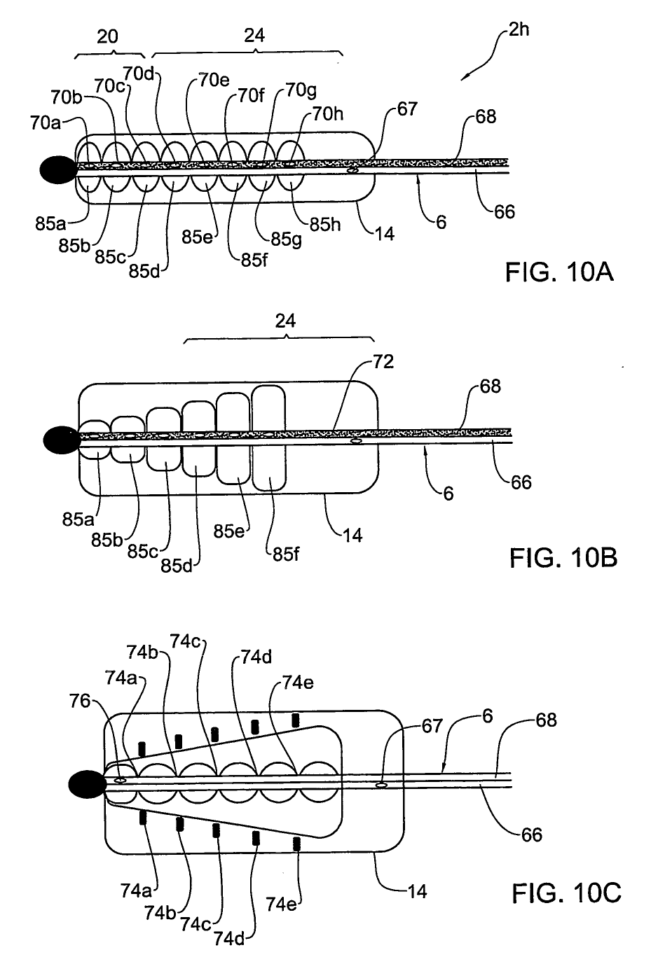

FIG. 10 shows the distal end 10 of a catheter 2h that may be used in the 30 system 1 for the catheter 2. The catheter 2h has several elements in common with the previously described embodiments of the catheter 2 and similar elements are identified by the same reference numeral without further comment.

In the catheter 2h, the shaft 6 has two lumens 66 and 68. The lumen 68 delivers pressurized fluid to each of a plurality of inner balloons 85 located in the interior of the balloon 14. 8 inner balloons 85a to 85h are shown in FIG. 10. This is by way of example only, and the catheter 2h may have any number of inner balloons 85 as required in any application. Each of the inner balloons 85a to 85h receives pressurized fluid from the lumen 68 via the holes 70 to 70h, respectively. At least the most anterior balloon (85a) is located in the anterior portion 20 of the balloon 14 so that the anterior portion 20 may be inflated prior to inflation of the posterior portion 24. The dimensions (diameter and/or length) of the inner balloons 85 may be selected as required in any application. In FIG. 10a, the inner balloons 85 are identical in size and shape. In FIG. 10b, the balloons 85 have equal longitudinal lengths but form a conical shape when inflated that tapers towards the distal end 10 of the catheter 2h. The inflation of the balloons 85 may occur simultaneously via the lumen 68 or sequentially via a single lumen in the shaft but with a fluid conducting system that selectively permits sequential filling of the internal balloons 85. As shown in FIG. 10b, the fluid conducting mechanism may comprise, for example, a rod 72 inserted into the lumen 68. Initially, the rod extends to the distal end of the catheter 2h, so as to block all of the holes 70 (FIG. 10b). As the rod 72 is withdrawn proximally in the lumen 68, the holes 70a to 70h are sequentially opened so as to allow sequential filling of the balloon s 85a to 85h. Alternatively, a single balloon 85 may be attached to the shaft 6 intermittently with bands 74 that divide the balloon 85 into compartments 78. Fluid is delivered via a single hole 76 in the wall of the shaft 6. When the pressure in the compartment 78a exceeds a predetermined pressure, the band 74a ruptures allowing inflation of the two most anterior compartments 78a and 78b. This process continues with the sequential rupturing of the bands 74b to 74g, until the inner balloon is completely inflated. After inflation of the inner balloon or balloons 85, the posterior portion 24 of the balloon 14 is inflated by delivering pressurized fluid to the balloon 16 via the lumen 66 and a hole 67. Inflation of the anterior portion 20 thus occurs prior to inflation of the posterior portion.

The catheter 2h may be used to expand a body passageway when a differential dilatation is needed at different parts of the passageway.

### Eighth Embodiment

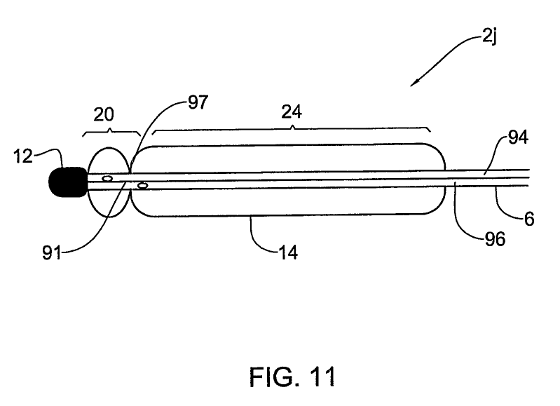

FIG. 11 shows the distal end of a catheter 2j that may be used in the system 1 for the catheter 2. The catheter 2j has several elements in common with the previously described embodiments of the catheter 2 and similar elements are identified by the same reference numeral without further comment.

In the catheter 2j, the shaft 6 has two lumens 94 and 96 which deliver pressurized fluid from the control unit 4 to the balloon 14. A shallow circular groove 91 is formed in the shaft 6. The groove 91 accommodates a ring 84 surrounding the balloon 14. The ring 84 separates the anterior portion 20 of the balloon 14 and the posterior portion 24 of the balloon 14. The ring 84 fits tightly over the balloon 14 so that fluid cannot flow between the anterior and posterior portions of the balloon 14. The anterior portion 20 of the balloon 24 is inflatable by means of the lumen 9 and hole 97a, while the posterior portion 24 is inflatable by means of the lumen 96 and the hole 97b. The anterior and posterior portions 20 and 24 are thus individually inflatable. Inflation of the anterior portion 20 thus occurs prior to inflation of the posterior portion.

### Ninth Embodiment

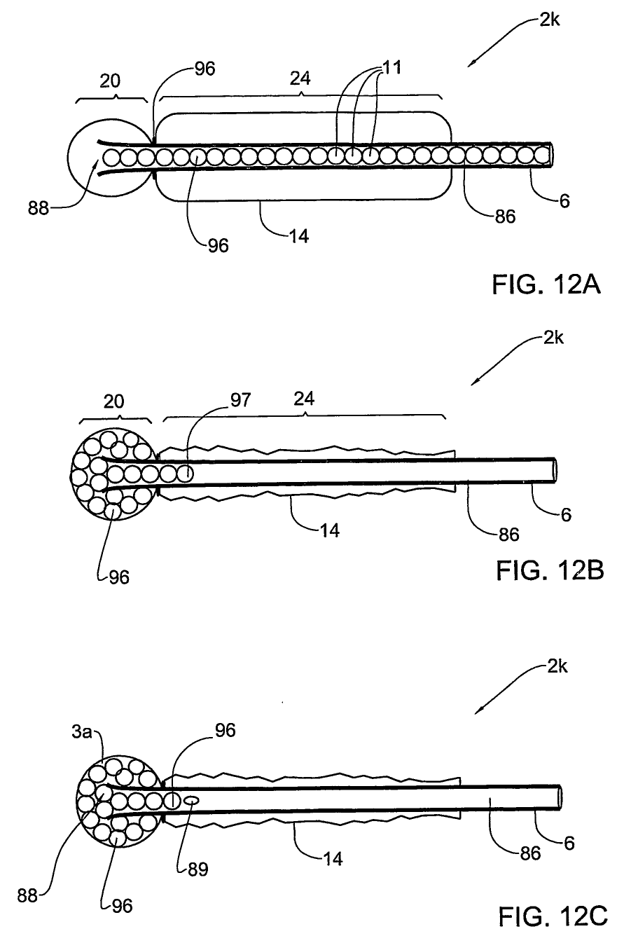

FIG. 12 shows the distal end of a catheter 2k that may be used in the system 1 for the catheter 2. The catheter 2k has elements in common with the previously described embodiments for the catheter 2, and similar components are identified by the same reference numeral without further comment.

In the catheter 2k, the shaft 6 has a single lumen 86 that conducts the pressure of fluid from the control unit 4 to the balloon 14. A first hole 88 is located at the tip of the shaft 6 that is larger than a second hole 89 located in the wall of the shaft 6. A shallow groove is formed in the wall of the shaft 6 between the holes 88 and 89. A ring 96 separates the anterior portion 20 and the posterior portion 24 the balloon 14. The posterior portion 24 may be inflated via the lumen 86 only through the hole 89. Due to the presence of the ring 96, no fluid can pass between the anterior and posterior portions of the balloon 14. As shown in FIG. 12a, initially, a plurality of spherical beads 96 are located inside the lumen 86. The beads 96 are made from a compressible material such as rubber. The diameter of the uncompressed beads 96 is slightly larger than the inner diameter of the lumen 86. Therefore, when the beads 96 are inserted into the lumen 86 the beads 96 are somewhat compressed so that the lumen 86 is blocked so as to prevent the flow of the pressurized fluid in the distal end of the lumen 86 and thus preventing inflation of either the anterior portion of the posterior portion. The anterior portion 20 is inflated by introducing the spheres 96 into the anterior portion 20 of the balloon 14 by delivering the pressurized fluid into the lumen 86 so as to ram the spheres 96 distally into the anterior portion 20, as shown in FIG. 12b. The number of the beads 96 is selected to be slightly more than the number of beads required to completely fill and inflate the anterior portion 20.

Fluid delivery is stopped when the last bead 97 is located just proximally to the hole 89 so that the posterior portion 24 cannot be inflated as the hole 89 is blocked. When it is desired to inflate the posterior portion 24, an additional volume of the pressurized fluid is delivered into the lumen 86 to push the bead 97 distally beyond the hole 89, as shown in FIG. 12c, so as to allow inflation of the posterior portion 24. Inflation of the anterior portion 20 thus occurs prior to inflation of the posterior portion 24. The margins of hole 88 diverge as shown in FIG. 12 to allow recovery of the beads 96 from the anterior portion 20 back into the lumen 86 when needed.

In the catheter 2k, the beads 96 may be replaced by a cord formed from a compressible material having a diameter slightly larger than the diameter of the lumen 86. The length of the cord is determined so that the cord has a volume slightly larger than the volume needed to inflate the anterior portion 20. A plunger may be used instead of the pressurized fluid to push the cord from the lumen 86 into the anterior portion 20 (not shown).

In the catheter 2k, the beads 96 may be replaced with a colloidal suspension of particulate material. In this case the hole 89 is replaced with a plurality of pores small enough to prevent passage to the colloidal particles through the wall of the shaft. A plunger may be used to deliver the suspension from the lumen 86 to the anterior portion 20 (not shown).

### Tenth Embodiment

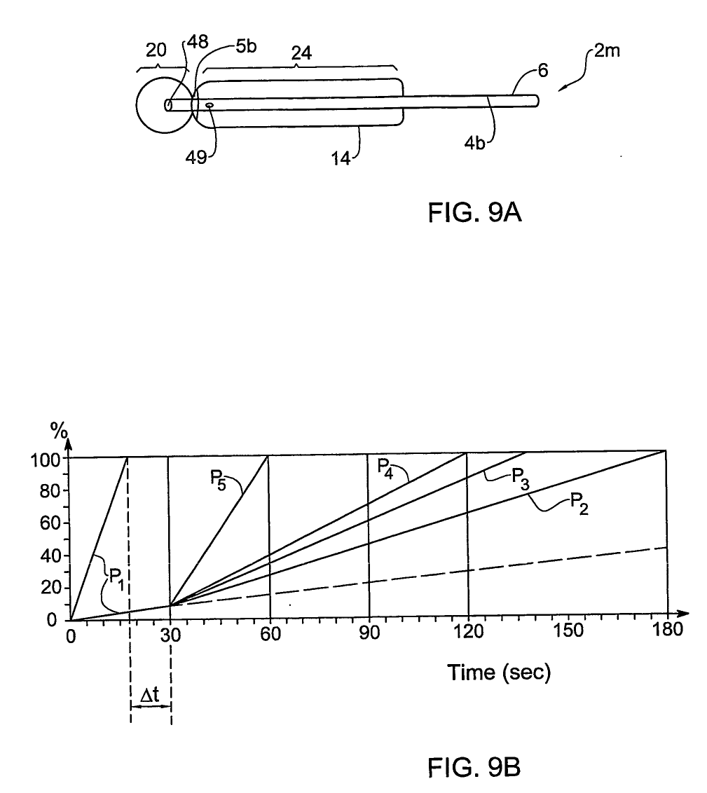

FIG. 9a shows the distal end of a catheter 2m that may be used in the system 1 for the catheter 2. The catheter 2m has several elements in common with previously described embodiments of the catheter 2, and similar elements are identified by the same reference numeral without further comment.

In the catheter 2m, the shaft 6 has a single lumen 46. Pressurized fluid is delivered to the anterior portion 20 via a hole 48 at the end of the shaft 6 and is delivered to the posterior portion 24 via a hole 49. The anterior and posterior portions are completely separated by a ring 56. When the filling process starts, pressurized fluid is delivered to the distal end of the lumen 46. The hole 48 is larger than the hole 49. The anterior chamber 20 is thus inflated faster than the posterior chamber 24. Moreover, because the volume of the anterior portion 20 is much smaller than that of the posterior chamber, the difference of the filling times of the anterior and posterior portions is sufficiently large that the anterior portion 20 is essentially inflated prior to inflation of the posterior portion 24. The differential of the inflation rates of the two compartments is described in the graph shown in FIG. 9b. The initial filling pressure in FIG. 9b is relatively low (i.e. the pressure P₁) until the anterior portion 20 is fully inflated. The filling pressure is then increased (e.g. any one of the pressures P₂, P₃, P₄, P₅ in FIG. 9b) in order to accelerate the inflation of the posterior portion 24.

### Eleventh Embodiment

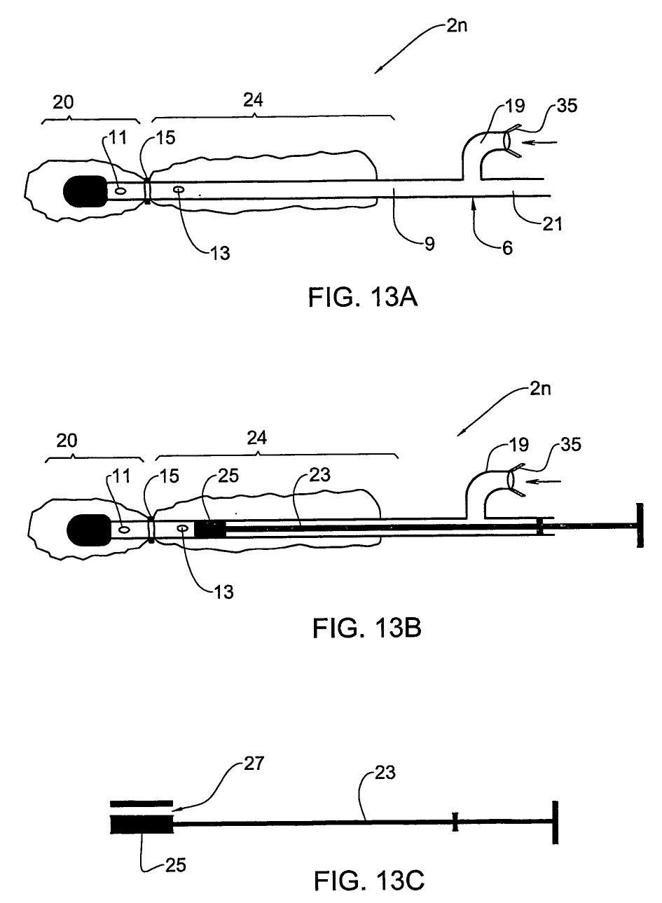

FIG. 13 shows a catheter 2n that may be used in the system 1 for the catheter 2. The catheter 2n has several elements in common with previously described embodiments of the catheter 2 and similar components are identified with the same reference numeral without further comment.

In the catheter 2n, the shaft 6 has a single lumen 9. The anterior portion 20 and the posterior portion 24 of the balloon 24 are separated by a ring 15, so that the pressurized fluid cannot flow between the anterior and posterior portions. The pressurized fluid is delivered to the anterior portion 20 and the posterior portion 24 from the lumen 9 via holes 11 and 13, respectively.

At the proximal end of the shaft 6 are two openings. A side opening 19 is provided with the socket 35 for attachment to the socket 33 of the control unit 4. An end opening 21 receives a plunger 23, as shown in FIG. 13b. The plunger 23 as a bulge 25 at its distal end. The bulge 25 has a canal 27 passing through it FIG. 13c).

The rod 23 is initially positioned so that the bulge 25 covers and blocks the hole 13. Pressurized fluid is delivered to the anterior portion 20 via the hole 11 and the canal 27. After the anterior portion 20 has been inflated, the piston 23 is pulled back so as to uncover the hole 13 so that the pressurized fluid can pass via the canal 27 and the hole 13 into the posterior portion 24 so as to inflate the posterior portion 24. Inflation of the anterior portion 20 thus occurs prior to inflation of the posterior portion 24.

Twelfth Embodiment

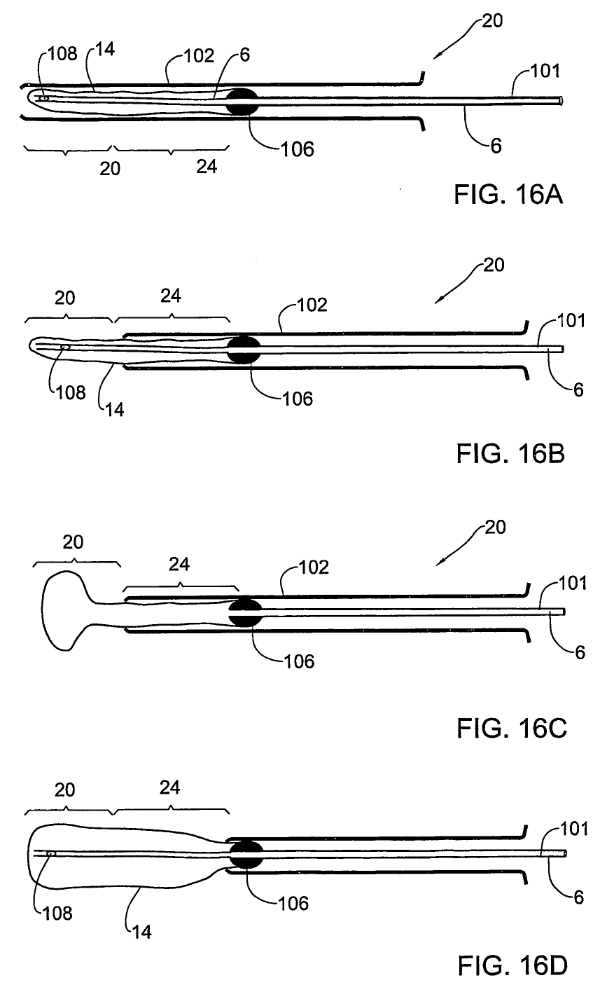

FIG. 16 shows a catheter 2o that may be used in the system 1 for the catheter 2. The catheter 2o has several components in common with previously described embodiments of the catheter 2, and similar elements are identified with the same reference numeral without further comment.

As shown in FIG. 16a, the balloon 14 is initially enfolded and wrapped around the shaft 6 which has a single lumen 101. The balloon 14 is located in a lumen 100 of an outer sleeve 102. The shaft 6 has a bulb 106 that fills the lumen of the sleeve 102 but enables relative movement of the shaft 6 and the outer sleeve 102. The pressurized fluid is delivered from the control-unit 4 through the lumen 101 via a hole 108 in the shaft 6 to the balloon 14.

In order to inflate the anterior portion 20 of the balloon 14, the outer sleeve 102 is pulled proximally relative to the shaft 6 so as to cause only the 5 anterior portion 20 of the balloon 14 to be exposed at the distal end of the catheter 20, as shown in FIG. 16b. In this configuration, delivery of the pressurized fluid will inflate only the exposed anterior portion 20, while the posterior portion 24 is prevented from inflating due to the presence of the sleeve 102 (FIG. 16c).

In order to inflate the posterior portion 24 of the balloon 14, the sleeve 102 is pulled proximally relative to the shaft 6 so as to expose the posterior portion 24. As the pressurized fluid continues to be delivered to the balloon 14 in this configuration, the posterior portion 24 inflates, as shown in FIG. 16d. Inflation of the anterior portion 20 thus occurs prior to inflation of the posterior portion.

### Thirteenth Embodiment

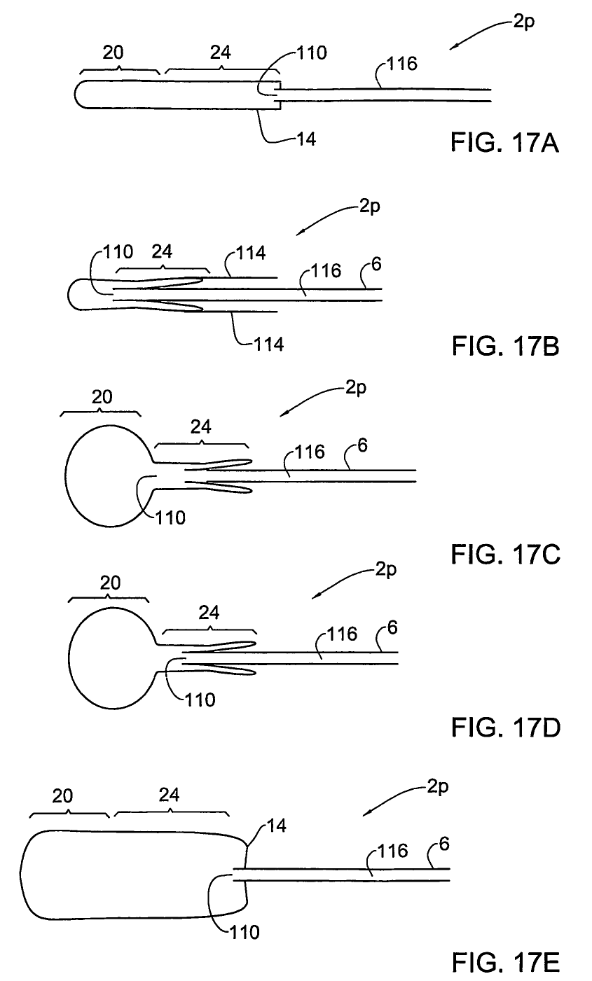

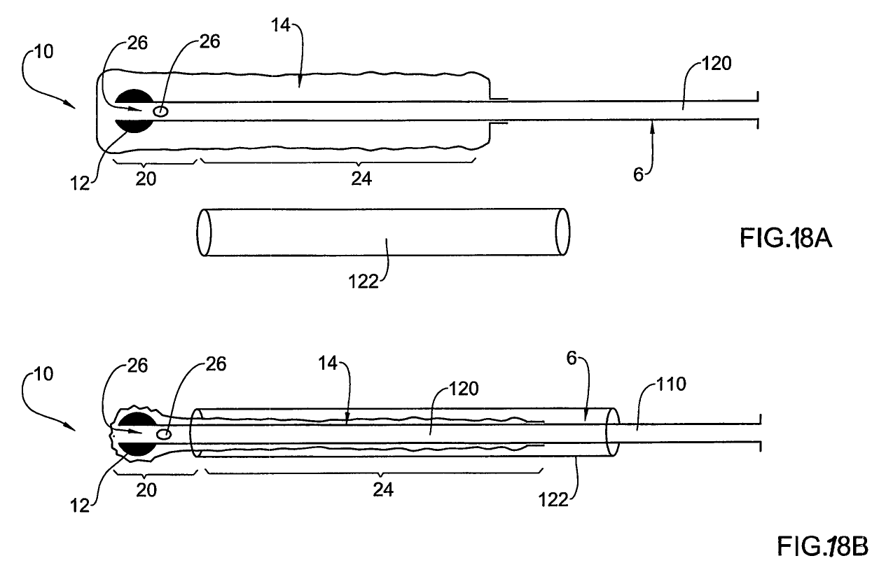

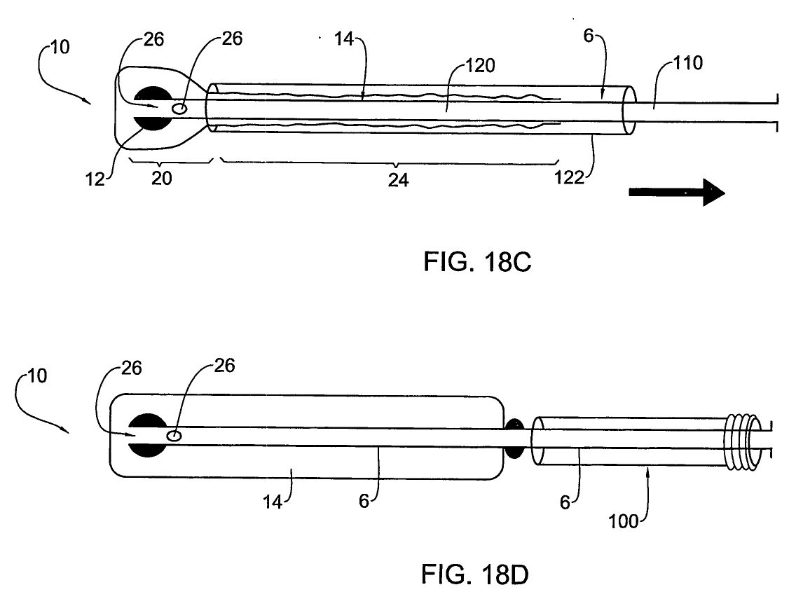

FIG. 17 shows a catheter 2p that may be used in the system 1 for the catheter 2. The catheter 2p has several components in common with previously described embodiments of the catheter 2, and similar elements are identified with the same reference numeral without further comment.

In the catheter 2p, the shaft 6 has a single lumen 116. As shown in FIG. 17a, the pressurized fluid enters the balloon 14 from a hole 110 located at the distal end of the shaft 6 and the proximal end of the balloon 14. The balloon 14 is folded onto the shaft 6 by pushing the shaft proximally into the balloon 14 so that the distal end of the shaft 6 enters the anterior portion 20, as shown in FIG. 17b. In this configuration, the anterior portion 24 is folded on itself, while the proximal portion 20 extends beyond the distal end of the shaft 6. An external sleeve 114 may be used to maintain the balloon 14 in this configuration during insertion of the balloon 14 into the body passageway.

After insertion of the balloon 14 into the passageway, the sleeve 114, if present, is withdrawn proximally. The anterior portion 20 is then inflated by delivering the pressurized fluid to the anterior portion 20 through the lumen 116 of the of the shaft 6 via the hole 110, as shown in FIG. 17c. The shaft 6 is then withdrawn proximally so as to bring the distal end of the shaft 6 into the posterior portion 24, as shown in FIG. 17d. Pressurized fluid is then delivered to the posterior portion 24 via the hole 110 as shown in FIG. 17e. Inflation of the anterior portion 20 thus occurs prior to inflation of the posterior portion.

### Fourteenth Embodiment

FIG. 18 shows a catheter 2q that may be used in the system 1 for the catheter 2. The catheter 2q has several components in common with previously described embodiments of the catheter 2, and similar elements are identified with the same reference numeral without further comment.

In the catheter 2q, the shaft 6 has a single lumen 120 that delivers the pressurized fluid to the balloon 14 via one or more holes 26, the balloon 14 is initially folded around the shaft 6. The posterior portion 24 is contained inside a cylindrical sleeve 122. The sleeve 120 is made from an inelastic flexible material, and may be made from the same material as the balloon. The sleeve 120 has a final diameter that is sufficiently large to contain the enfolded posterior portion 24 of the balloon 14. The sleeve 120 cannot be stretched beyond the final predetermined diameter. Therefore, when the posterior portion 24 of the balloon 14 is located inside the sleeve, its inflation is strictly limited by the final diameter of the sleeve 120. The anterior portion 20 can be inflated without any limitation imposed by the sleeve, as shown in FIG. 18c. After the anterior portion 20 is inflated the sleeve 120 is withdrawn proximally, as shown in FIG. 18d. The posterior portion 24 of the balloon 14 is now freed from the sleeve 120 and maybe inflated without any limitation other than resistance of the passage, and the final predetermined diameter of the balloon 14, as shown in FIG. 18d.

1. A catheter for expanding a body passageway leading to a body cavity, comprising:
(a) an elongated shaft having a proximal end and a distal end, a wall and at least one lumen; and
(b) an inflatable first balloon located at the distal end of the shaft having an anterior portion and a posterior portion;
wherein the anterior portion is inflatable prior to inflation of the posterior portion.

2. The catheter according to claim 1 wherein the shaft has one lumen.

3. The catheter according to claim 1 wherein the shaft has two lumens.

4. The catheter according to claim 1 wherein the anterior and posterior portions of the first balloon are separated by a ring surrounding the balloon.

5. The catheter according to claim 4 wherein the ring prevents passage of fluid between the anterior portion and the posterior portion when a fluid pressure in the anterior portions is below a predetermined pressure.

6. The catheter according to claim 5 wherein the shaft has a single lumen and a hole in the wall of the shaft for inflating the anterior portion.

7. The catheter according to claim 5 wherein the ring expands when the fluid pressure in the anterior pressure is above the predetermined pressure.

8. The catheter according to claim 5 wherein the ring slides distally over the first balloon when the fluid pressure in the anterior pressure is above the predetermined pressure.

9. The catheter according to claim 5 wherein the ring is formed from a cord wrapped around the first balloon, the cord being detachable from the first balloon by pulling on an end of the cord.

10. The catheter according to claim 1 comprising a second balloon located in the anterior portion of an interior of the first balloon.

11. The catheter according to claim 10 wherein the shaft has a first lumen in fluid communication with the interior of the second balloon and a second lumen in fluid communication with the interior of the posterior portion of the balloon.

12. The catheter according to claim 10 wherein the shaft has a lumen in fluid communication with the second balloon, the second balloon bursting when a fluid pressure in the second balloon exceeds a predetermined pressure.

13. The catheter according to claim 1 comprising a second balloon and a third balloon, the second and third balloons being located in the interior of the first balloon, wherein the shaft has a first lumen in fluid communication with the interior of the second balloon, a second lumen in fluid communication with the third balloon, and a third lumen in fluid communication with the posterior portion of the first balloon.

14. The catheter according to claim 1 comprising a plurality of inner balloons located in the interior of the first balloon, at least an anterior most of the inner balloons being located in the anterior portion of the first balloon, the shaft having a first lumen in fluid communication with each of the inner balloons and having a second lumen in fluid communication with the posterior portion of the first balloon.

15. The catheter according to claim 14 further comprising a rod dimensioned to be slidable in the first lumen and to prevent passage of fluid from the first lumen into one or more of the inner balloons.

16. The catheter according to claim 1 comprising a second balloon in the interior of the first balloon, at least a portion of the second balloon being located in the anterior portion of the first balloon, the second balloon being divided into compartments by bands surrounding the second balloon, the shaft having a lumen in fluid communication with an anterior most compartment.

17. The catheter according to claim 16 wherein a band bursts when a fluid pressure in a compartment adjacent to the band is greater than a predetermined fluid pressure.

18. The catheter according to claim 1 wherein the shaft has a lumen having a first hole in the anterior portion and a second hole in the posterior portion, and further comprising beads dimensioned to fit in the lumen and to pass through the first hole and not to pass through the second hole, the beads inflating the anterior portion when introduced into the anterior portion.

19. The catheter according to claim 18 further comprising a rod for ramming the beads into the anterior portion.

20. The catheter according to claim 18 wherein a bead in the lumen in contact with the second hole blocks the second hole preventing flow of fluid from the lumen into the posterior portion.

21. The catheter according to claim 1 wherein the shaft has a lumen having a first hole in the anterior portion and a second hole in the posterior portion, and further comprising a cord dimensioned to fit in the lumen and to pass through the first hole and not to pass through the second hole, the cord inflating the anterior portion when introduced into the anterior portion.

22. The catheter according to claim 21 further comprising a rod for ramming the cord into the anterior portion.

23. The catheter according to claim 21 wherein the cord blocks the second hole preventing flow of fluid from the lumen into the posterior portion.

24. The catheter according to claim 1 wherein the shaft has a lumen having a first opening in the anterior portion and a second opening in the posterior portion, the first opening being larger than the second opening.

25. The catheter according to claim 1 wherein the anterior portion and the second portion are separated by a ring preventing flow of fluid between the anterior portion and the posterior portion, and wherein the shaft has a lumen having a first hole in the anterior portion and a hole in the posterior portion, the catheter further comprising a rod having a piston slidable in the lumen, the piston having a canal for transfer of fluid through the piston, the piston preventing flow of fluid from the lumen into the posterior portion when the piston covers the second hole.

26. The catheter according to claim 1 having an outer sleeve slidable from a first position in which the posterior portion of the balloon is contained in the sleeve and a second position in which the posterior portion of the balloon is not contained in the sleeve.

27. The catheter according to claim 26 wherein the sleeve is made from an inelastic flexible material.

28. The catheter according to claim 1 wherein the shaft has a single lumen and the shaft terminates at the distal end of the balloon.

29. The catheter according to claim 1 wherein the body passage is a cervix.

30. A system for inflating a body passageway leading to a body cavity, comprising:
(a) A catheter according to claim 1 having a first socket on the proximal end of the shaft; and
(b) A control unit having a second socket mating with the first socket; the control unit providing pressurized fluid to a lumen of the shaft when the first socket is attached t the second socket.

31. A method for dilating a body passageway leading to a body cavity, comprising:
(a) inserting the distal end of a catheter according to claim 1 into the passageway with the first balloon in an uninflated state;
(b) positioning the anterior portion of the first balloon inside the body cavity;
(c) inflating the anterior portion of the first balloon in the body cavity;
(d) displacing the distal end proximally so as to lodge the inflated anterior portion in an opening of the passageway into the body cavity; and
(e) inflating the posterior portion in the passageway so as to expand the passageway.

32. A method for dilating a body passageway leading to a body cavity, comprising:
(a) delivering the anterior portion of a catheter according claim 1 to a segment of the passageway with the first balloon in an uninflated state;
(b) inflating the anterior portion of the first balloon in the segment;
(c) displacing the distal end proximally;
(d) inflating the posterior portion in the passageway; and
(e) repeating steps (b) to (d) as required to achieve a desired dilation of the body passageway.

33. A method for determining whether the resistance of a wall of a body passageway at a first location in the passageway is normal comprising:
(a) inserting a distal end of a catheter according to claim 1 into the passageway;
(b) positioning the first balloon at the first location in the passageway;
(c) inflating the balloon; and
(d) determining whether an external pressure exerted by the passageway wall on the inflated balloon is above a predetermined threshold;
an external force exerted on the balloon above the predetermined threshold being indicative of a normal resistance.
